(19) United States
(12) Patent Application Publication
(10) Pub. No.: US 2015/0011503 A1
(43) Pub. Date: Jan. 8, 2015

(54) HEMOSTATIC DRESSING AND METHOD FOR MANUFACTURING THE SAME

(71) Applicant: BENQ MATERIALS CORPORATION, Taoyuan County (TW)

(72) Inventor: KUO-YUNG CHANG, Hsinchu County (TW)

(73) Assignee: BENQ MATERIALS CORPORATION, Taoyuan County (TW)

(21) Appl. No.: 14/275,277

(22) Filed: May 12, 2014

(30) Foreign Application Priority Data
Jul. 4, 2013 (TW) 102123969

(51) Int. Cl. A61L 26/00

(52) U.S. Cl. CPC A61L 26/0023
USPC 514/55

(57) ABSTRACT

A hemostatic dressing and method for manufacturing the same are disclosed. The hemostatic dressing comprises 25%-50% by weight of acidic chitosan fiber and 50%-75% by weight of crosslinked chitosan matrix. The hemostatic dressing of the present invention has better hemostasis effect and can maintain high stress intensity when be used.

### CROSS-REFERENCE TO RELATED APPLICATION

This application claims the benefit under 35 U.S.C. §119 of Taiwanese Patent Application No. 102123969, filed Jul. 4, 2013, which is hereby incorporated by reference in its entirety.

### BACKGROUND OF THE INVENTION

1. Field of the Invention

The present invention relates to a hemostatic dressing. More particularly, the present invention relates to a hemostatic dressing comprising chitosan fiber. The hemostatic dressing has a better hemostasis effect and can maintain high stress intensity when be used.

2. Description of the Related Art

Chitosan is a polymer produced by deacetylation of chitin which is a nature polymer extracted from organism and mainly found in crustaceans (e.g., shrimps and crabs), exoskeleton of insects and walls of fungi. Chitosan is a linear polysaccharide composed of distributed β-(1-4)-linked D-glucosamine and N-acetyl-D-glucosamine. Chitosan has good biocompatibility, biological activity, noncytotoxicity and biodegradation in cells, thus can be used in biomedical applications. After mixed with organic acid, chitosan will have positive charge, and lead to involvement of platelets and rapidly stop bleeding therefore.

In this regard, wound dressing composed from chitosan nonwoven has been used in clinical application. However, chitosan nonwoven wound dress has a disadvantage of low stress intensity, chitosan nonwoven wound dress will rapidly become gelatinous and break after contacting blood. Some methods for increasing stress intensity of chitosan nonwoven wound dress are provided in the related art, such as using rayon which is insoluble in water to maintain the stress of chitosan nonwoven wound dress disclosed in U.S. Pat. No. 7,981,872; a nonwoven wound dress composed from chitosan fiber and cellulose fiber, cellulose fiber is insoluble in water and is used to maintain the stress intensity when the wound dress is used in wound of patient disclosed in U.S. Pat Pub No. 2011/0236433 describes. However, composing another material which is insoluble in water can maintain the stress of wound dress, but meanwhile cause the ratio of chitosan fiber decrease and thus hemostasis effect of wound dress will be affected thereof. Therefore, to increase the ration of chitosan fiber, and meanwhile maintain the stress intensity when be used is still a problem to be solved.

The aforesaid disadvantages of the prior art can be alleviated. Therefore, the present invention is to provide a novel hemostatic dress to improve the disadvantages mentioned above. The hemostatic dress comprising acidic chitosan fiber and crosslinked chitosan matrix has higher amino content and thus leading to involvement of platelets and rapidly stops bleeding. The hemostatic dress of the present invention has better hemostasis effect and high stress intensity and can be used to stop bleeding and dressing wound.

### BRIEF SUMMARY

The present invention is to provide a hemostatic dressing which has better hemostasis effect and can maintain high stress intensity when be used.

According to an aspect of the present invention, a hemostatic dressing is provided.

In an embodiment of the present invention, the hemostatic dressing comprises 25%-50% by weight of acidic chitosan fiber and 50%-75% by weight of crosslinked chitosan matrix.

In an embodiment of the present invention, a wet tensile of the hemostatic dressing is in the range of from 200 gf to 600 gf and a dry tensile of the hemostatic dressing is in the range of from 1000 gf to 2600 gf.

In an embodiment of the present invention, an amino content of the hemostatic dressing is in the range of from 50% to 90%.

According to another aspect of the present invention, a method for manufacturing hemostatic dressing is provided.

In an embodiment of the method of the present invention, the method of manufacturing hemostatic dressing, comprising the steps of:

(a) providing a chitosan fiber;

(b) adding 25%-50% by weight of the chitosan fiber in an acidic organic solvent;

(c) conducting a dehydration and drying treatment to form an acidic chitosan fiber;

(d) mixing 50%-75% by weight of the chitosan fiber and a crosslinking agent in an organic solvent and forming a crosslinked chitosan matrix after a polymerization reaction;

(e) conducting a dehydration and drying treatment to the crosslinked chitosan matrix; and

(f) needle punching a mixture of the acidic chitosan fiber and the crosslinked chitosan matrix to form a chitosan nonwoven.

In an embodiment of the method of the present invention, the chitosan fiber has a degree of deacetylation between 80 and 99%.

In an embodiment of the method of the present invention, the acidic organic solvent comprises phosphoric acid, citric acid, malic acid, succinic acid, acetic acid, hydrochloric acid or lactic acid.

In an embodiment of the method of the present invention, a pH value of the acidic organic solvent is in a range of from 2 to 6.

In an embodiment of the method of the present invention, the organic solvent is select from a group consisting of methanol, ethanol, acetone and isopropanol.

In an embodiment of the method of the present invention, the crosslinking agent is select from a group consisting of glutaraldehyde, formaldehyde, acetaldehyde and ethylene isopropanol.

In an embodiment of the method of the present invention, a time of the polymerization reaction is in the range of from 60 minutes to 180 minutes.

The above and other aspects of the invention will become better understood with regard to the following detailed description of the preferred but non-limiting embodiment(s).

### BRIEF DESCRIPTION OF THE DRAWINGS

FIG. 1 is a flow chart of a method for manufacturing a hemostatic dressing, of a preferred embodiment of the present invention.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

According to an aspect of the present invention, a hemostatic dressing is provided. The hemostatic dressing has more amino content, thus the hemostatic dressing has better hemostasis effect. Moreover, the hemostatic dressing can maintain high stress intensity when be used.

The hemostatic dressing of the present invention comprises 25%-50% by weight of acidic chitosan fiber and 50%-75% by weight of crosslinked chitosan matrix.

In an embodiment of the present invention, a dry tensile of the hemostatic dressing is in the range of from 1000 gf to 2600 gf in dry condition, a wet tensile of the hemostatic dressing is in the range of from 200 gf to 600 gf in wet condition. A liquid absorption rate of the hemostatic dressing is in the range of from 14% to 18%. And an amino content of the hemostatic dressing is in the range of from 50% to 90%.

According to another aspect of the present invention, a method for manufacturing hemostatic dressing is provided. The hemostatic dressing manufactured by the method of the present invention has better hemostasis effect. Moreover, the hemostatic dressing can maintain high stress intensity when be used.

The above method comprises but not limited to the following steps.

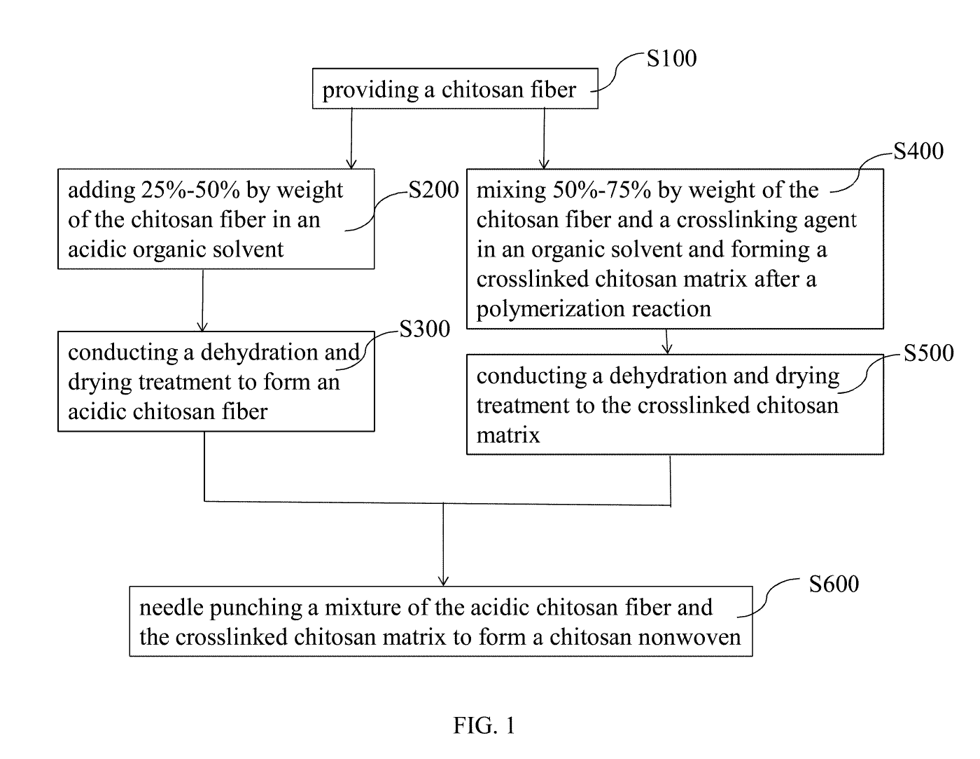

A preferred embodiment of the method of the present invention is illustrated by FIG. 1. FIG. 1 is a flow chart of a method for manufacturing a hemostatic dressing of a preferred embodiment of the present invention. Firstly, in step S100, a chitosan fiber is provided. In a preferred embodiment of the method of the present invention, the chitosan fiber has a degree of deacetylation between 80 and 99%.

In step S200, 25%-50% by weight of the chitosan fiber is added in an acidic organic solvent. In this step, the acidic organic solvent can be stirred by a stirring machine selectively. In an embodiment of the method of the present invention, a time of stirring is about 2 hours. The acidic organic solvent comprises phosphoric acid, citric acid, malic acid, succinic acid, acetic acid, hydrochloric acid or lactic acid. And a pH value of the acidic organic solvent is in a range of from 2 to 6, more preferably in a range of from 3.5 to 5.5. In an embodiment of the method of the present invention, the acidic organic solvent comprises acetic acid, and a pH value of the acidic organic solvent is about 4.7.

Then, in step S300, dehydration and drying treatment are conducted to form an acidic chitosan fiber. The drying treatment is conducted at a temperature in the range between about 70° C. to about 80° C. and a time of drying treatment is in the range between 160 minutes to 200 minutes. In an embodiment of the method of the present invention, the drying treatment is conducted at 75° C. , and a time of drying treatment is about 180 minutes.

At the same time, 50%-75% by weight of the chitosan fiber and a crosslinking agent are mixed in an organic solvent and a crosslinked chitosan matrix is formed after a polymerization reaction as shown in step S400. A time of the polymerization reaction is in the range of from 60 minutes to 180 minutes. In this step, the organic solvent can be stirred by a stirring machine selectively. In an embodiment of the method of the present invention, and a time of the polymerization reaction is about 120 minutes.

Suitable organic solvent can be but not limited to methanol, ethanol, acetone or isopropanol. Suitable crosslinking agent can be but not limited to glutaraldehyde, formaldehyde, acetaldehyde or ethylene isopropanol. In an embodiment of the method of the present invention, the organic solvent is ethanol and the crosslinking agent is glutaraldehyde.

Then, in step S500, dehydration and drying treatment are conducted to the crosslinked chitosan matrix. In an embodiment of the method of the present invention, the drying treatment is conducted at 75° C. , and a time of drying treatment is about 180 minutes.

Finally, a mixture of the acidic chitosan fiber and the crosslinked chitosan matrix are needle punched to form a chitosan nonwoven as shown in step S600.

The above mentioned needle punching is a well known method for manufacturing nonwoven. Needles penetrate in fluffy chitosan fiber web after the drying treatment, some chitosan fiber of the web is carried by the barbs on the needle and moved when the needle through the web, meanwhile, the web is compressed due to friction. After the needles penetrate in the web with a depth, the needle move up and the chitosan fiber carried by the barbs is kept at the surface of web vertically from the barbs, thus chitosan fiber can compress tightly with each other to form a chitosan nonwoven. In an embodiment of the present invention, the chitosan nonwoven comprises 25% by weight of acidic chitosan fiber and 75% by weight of crosslinked chitosan matrix. In another embodiment of the present invention, the chitosan nonwoven comprises 50% by weight of acidic chitosan fiber and 50% by weight of crosslinked chitosan matrix.

The hemostatic dressing manufactured by the method of the present invention has better hemostasis effect and can maintain high stress intensity when be used.

The present invention will be explained in further detail with reference to the examples. However, the present invention is not limited to these examples.

### EXAMPLE

### Preparation of Acidic Chitosan Fiber

1000 g of acetic acid and 19000 g of ethanol were mixed to form an acidic organic solvent (pH is about 4.7). 300 g of chitosan fiber (available from Hismer, China) was added into the acidic organic solvent. After stirred for 2 hours, the fiber was dehydrated for 2 minutes. Then, a heat treatment was conducted to the fiber at 75° C. for 180 minutes to obtain an acidic chitosan fiber.

### Preparation of Crosslinked Chitosan Matrix

300 g of chitosan fiber (available from Hismer, China) and 50 g of glutaraldehyde as crosslinking agent were added to 10000 g of 0.5% ethanol. After stirred for 2 hours, dehydration was conducted for 2 minutes. Then, a heat treatment was conducted to the fiber at 75° C. for 180 minutes to obtain an crosslinked chitosan matrix.

### Examples 1-2

### The Preparation of the Hemostatic Dressing

Acidic chitosan fiber and crosslinked chitosan matrix were needle punched at the amounts shown in following Table 1 to form a hemostatic nonwoven dressing.

TABLE 1
The composition of hemostatic dressing of Example 1-Example 2

Example (wt %)

| Composition | 1 | 2 |
|---|---|---|
| Acidic chitosan fiber | 50 | 29 |
| Crosslinked chitosan matrix | 50 | 71 |

The method of physical property tests of the hemostatic dressing are as following:

### Dry Tensile Test

The dry tensile of examples were measured by tension machine with 50 Kg load cell and tensile speed of 200 mm/min until hemostatic dressing was broken, then the maximum breaking tenacity was recorded.

### Wet Tensile Test

3 ml of saline was absorbed by hemostatic dressing, then the wet tensile was measured by the above mentioned condition, maximum breaking tenacity was recorded until hemostatic dressing was broken.

### Liquid Absorption Rate Test

A weight of dry hemostatic dressing (W1) was measured, and then the dry hemostatic dressing and saline which was 40 times the weight of hemostatic dressing were placed into 37° C. incubator for 30 minutes. A weight of wet hemostatic dressing (W2) was measured, and the liquid absorption rate was calculated according to following formula:

Liquid absorption rate=(W2−W1)/W1×100%

### Amino Content Test

0.05 g of toluidine blue and 49.95 g of 95% ethanol were fully dissolved in a beaker as indicator. Then, 0.5 g of hemostatic dressing was fully dissolved into 99.5 g of 5% acetic acid to form a chitosan solution. Then, 1 g of the chitosan solution and 30 g of water were fully mixed to form test solution. Six drops of the indicator were added into the test solution and the test solution became blue, and then the test solution was titrated with polyvinylsulfuric acid potassium salt (PVSK) until the test solution became purple. The amino content was calculated according to following formula:

$$Amino content ( \% ) = \frac{X}{\frac{161}{\frac{X}{161} + \frac{Y}{203}}} \times 100 \%$$

$$X = \frac{1}{400} \times \frac{1}{1000} \times f \times 161 \times V$$

$$Y = 0.5 \times \frac{1}{100} - X$$

$$V = Titration volume of \frac{1}{400} N PVSK ( ml )$$

$$f = Titer of PVSK$$

### Platelet Aggregation Test

50 ml of blood was exsanguinated from ear vein of New Zealand white rabbits, platelet suspension was obtained after separated by centrifugal purification. 0.6 ml of platelet suspension and magnet for accelerating reaction were added into tube, and then 1 mg of hemostatic dressing was placed in the up part of tube and soaked into platelet suspension. After reacted for 20 minutes, the tube was placed into platelet aggregation analyzer to analyzing coagulation by turbidimetry.

The results of physical property tests of Example 1-Example 2 of the present invention were shown as the following Table 2.

TABLE 2
The result of physical property tests of Example 1-Example 2

Example

| Item | 1 | 2 |
|---|---|---|
| Dry tensile (gf) | 2020 | 1129 |
| Wet tensile (gf) | 249 | 426 |
| Liquid absorption rate (g/g) | 17.4 | 17.1 |
| Amino content (%) | 85.3 | 69.5 |
| Platelet aggregation rate (%) | 87 | 68 |

As shown in Table 2, the hemostatic dressing according to the present invention has excellent liquid absorption rate, amino content and platelet aggregation rate, thus, the hemostatic dressing has better hemostasis effect. In addition, the hemostatic dressing according to the present invention has a dry tensile not less than 1129 gf when being dry, and a wet tensile not less than 249 gf when being wet.

From the forgoing, the hemostatic dressing according to the present invention has excellent hemostasis effect and good physical properties, especially can maintain high stress intensity when being wet.

While the invention has been described by way of example(s) and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A hemostatic dressing comprises 25%-50% by weight of acidic chitosan fiber and 50%-75% by weight of crosslinked chitosan matrix.

2. The hemostatic dressing according to claim 1, wherein a wet tensile of the hemostatic dressing is in the range of from 200 gf to 600 gf and a dry tensile of the hemostatic dressing is in the range of from 1000 gf to 2600 gf.

3. The hemostatic dressing according to claim 1, wherein an amino content of the hemostatic dressing is in the range of from 50% to 90%.

4. A method for manufacturing hemostatic dressing, comprising the steps of:
(a) providing a chitosan fiber;
(b) adding 25%-50% by weight of the chitosan fiber in an acidic organic solvent;
(c) conducting a dehydration and drying treatment to form an acidic chitosan fiber;
(d) mixing 50%-75% by weight of the chitosan fiber and a crosslinking agent in an organic solvent and forming a crosslinked chitosan matrix after a polymerization reaction;
(e) conducting a dehydration and drying treatment to the crosslinked chitosan matrix; and
(f) Needle punching a mixture of the acidic chitosan fiber and the crosslinked chitosan matrix to form a chitosan nonwoven.

5. The method of manufacturing hemostatic dressing hemostatic dressing according to claim 4, wherein the chitosan fiber has a degree of deacetylation between 80 and 99%.

6. The method of manufacturing hemostatic dressing hemostatic dressing according to claim 4, wherein the acidic organic solvent comprises phosphoric acid, citric acid, malic acid, succinic acid, acetic acid, hydrochloric acid or lactic acid.

7. The method of manufacturing hemostatic dressing hemostatic dressing according to claim 4, wherein a pH value of the acidic organic solvent is in a range of from 2 to 6.

8. The method of manufacturing hemostatic dressing hemostatic dressing according to claim 4, wherein the organic solvent is select from a group consisting of methanol, ethanol, acetone and isopropanol.

9. The method of manufacturing hemostatic dressing hemostatic dressing according to claim 4, wherein the crosslinking agent is select from a group consisting of glutaraldehyde, formaldehyde, acetaldehyde and ethylene isopropanol.

10. The method of manufacturing hemostatic dressing hemostatic dressing according to claim 4, wherein a time of the polymerization reaction is in the range of from 60 minutes to 180 minutes.
